(19) United States
(12) Patent Application Publication
(10) Pub. No.: US 2015/0316567 A1
(43) Pub. Date: Nov. 5, 2015

(54) METHODS FOR ASSESSING IMMUNOGENICITY

(71) Applicant: SANOFI PASTEUR LIMITED, Toronto (CA)

(72) Inventors: Marie-Danielle Salha, Toronto (CA); Scott Gallichan, Burlington (CA); Roger Brookes, Toronto (CA); Martina Ochs-Onolemhemhen, Lyon (FR)

(73) Assignee: SANOFI PASTEUR LIMITED, Toronto, ON (CA)

(21) Appl. No.: 14/651,709

(22) PCT Filed: Dec. 13, 2014
(86) PCT No.: PCT/CA2013/050964

§ 371 Date: Jun. 12, 2015

Related U.S. Application Data
(60) Provisional application No. 61/737,234, filed on Dec. 14, 2012.

(51) Int. Cl. G01N 33/94; G01N 33/68; C07K 7/08

(52) U.S. Cl. CPC G01N 33/94; C07K 7/08; G01N 33/6854

(57) ABSTRACT

This disclosure relates to methods for detecting the potency of a drug preparation and/or detecting antibodies in a host reactive against one or more antigens.

### FIELD OF THE DISCLOSURE

This disclosure relates to methods for detecting the potency of a drug product and/or detecting antibodies in a host reactive against one or more antigens.

### BACKGROUND OF THE DISCLOSURE

The potency of a drug product containing antigens (e.g., a vaccine) typically depends upon the presence of immunogenic antigens therein (an “intact” drug product). The quality of such drug products (e.g., whether or not it contains a sufficient quantity or quality of antigens) may be difficult to ascertain. Similarly, it is typically difficult to identify protective vaccines without conducting clinical trials. The availability of a simple, accurate in vitro assay would solve this problem.

Assay systems have been described that provide for the detection of antibodies to bacterial cell surface antigens on the cell surface of the microorganism. For instance, WO 2011/014947 (pub. Feb. 10, 2011 (corresponding to U.S. Ser. No. 13/388,042 filed Jul. 30, 2010)) describes a flow cytometric surface accessibility assay for measuring the accessibility of various P. gingivalis proteins on intact cells to mouse monoclonal antibodies raised against the corresponding recombinant proteins. Similarly, WO 2011/075823 A1 (pub. Jun. 30, 2011 (corresponding to U.S. Ser. No. 13/515,093 filed Dec. 20, 2010)) describes a flow cytometric surface accessibility assay for identifying the Streptococcus pneumoniae antigens PspA, PhtD and PcpA (alone or in combination) using human purified monoclonal antibodies (for PspA), purified rabbit antigen-specific antibodies (for PhtD and PcpA) and live bacteria. These assay systems were used to show that antibodies generated in animals against a purified recombinant protein or purified human monoclonal antibodies that are specific to that protein and could bind native and intact proteins on the surface of live bacteria and be detected by flow cytometry. This disclosure describes assay systems that improve upon known assay systems by including a competitive step such that antigen quantity and/or quality of a drug product, and/or the protective potential of a vaccine, may be ascertained. This disclosure also describes the surprising use of SASSY as a substitute for typical assay systems that measure vaccine immunogenicity and/or efficacy in human beings. Various embodiments of such assay systems are described herein.

### BRIEF DESCRIPTION OF THE DRAWINGS

FIG. 1. Graphical results of PcpA blocking study. Anti-PcpA polyclonal serum was generated by immunizing rabbits intramuscularly with 10 μg/dose and boosted twice with intact PcpA formulated with Phosphate treated AIOOH and used at 10% final concentration (using PBS as diluent) in a competitive SASSY as described herein. A diluted serum was preincubated for at least 1 hour at room temperature with a titration (1:2 dilution factor starting at 10 μg/mL) of either intact or stressed PcpA proteins following desorption of AIOOH.

FIG. 2. Graphical results of Competitive SASSY with anti-PcpA Monoclonal Antibodies. A pool of anti-PcpA monoclonal antibodies (1-12, 1-29, 2B3, 6A4, and 9G11) was used at a final concentration of 5 μg/mL each (using PBS as diluent) in a competitive SASSY as desribed herein. Diluted monoclonal antibodies were preincubated for at least 1 hour at room temperature with a titration (7:10 dilution factor starting at 10 μg/mL (final concentration)) of either intact or stressed PcpA proteins.

FIG. 3. Graphical results of Competitive SASSY using anti-PhtD Polyclonal Sera. Anti-PhtD polyclonal sera was generated by immunizing rabbits intramuscularly with intact PhtD formulation with phosphate treated AIOOH and used at 5% final concentration (using PBS as diluent) in a competitive SASSY as described herein. Diluted sera was preincubated for at least 1 hour at room temperature with a titration (7:10 dilution factor starting at 10 μg/mL (final concentration)) of either intact or stressed PhtD proteins.

FIG. 4. Grahical results of Competitive Sassy using anti-PhtD monoclonal antibodies. A pool of anti-PhtD monoclonal antibodies (3C, 4D5, 5B7, 6B7, 8G4 and 8H6) was used at a final concentration of 2.5 μg/mL each (using PBS as diluent) in a competitive SASSY as described herein. Diluted monoclonal antibodies were preincubated for at least 1 hour at room temperature with a titration (7:10 dilution factor starting at 10 μg/mL final concentration) of either intact or stressed PhtD proteins.

FIG. 5. Graphical results of titration of Sera from Rabbits Immunized with Intact or Stressed PcpA Formulations. Anti-PcpA sera was generated by immunizing rabbits with either intact or stressed PcpA formulations (1 or 6 weeks at 43-47° C.). Pooled sera from each group were titrated with a 1:2 dilution factor in SASSY as described herein.

FIG. 6. Bar graph depicting Survival of Mice Injected with Diluted anti-PcpA Sera from Rabbits Immunized with Intact or Stressed monovalent PcpA Formulations in Passive Protection. In two studies, anti-PcpA sera obtained from rabbits immunized with PcpA protein formulations that were intact, or stressed at 43-47° C. for 1 or 6 weeks, were tested in CBA/n mice in a passive protection model as described herein. Sera were diluted at the concentrations indicated below (PBS was used as a control) and used to passively immunize i p CBA/N mice, and 50 cfu of A66.1^{Mn-} were delivered i.v. 1 hour following passive immunization. Survival rates from both studies were combined and plotted.

FIG. 7. Graphical results of titration of Sera from Rabbits Immunized with Intact or Stressed PhtD Formulations. Anti-PhtD sera was generated by immunizing rabbits with either intact or stressed PhtD formulations (1 or 6 weeks at 43-47° C.). Pooled sera from each group were titrated with a 1:2 dilution factor in SASSY as described herein.

FIG. 8. Bar graph depicting Survival of Mice Injected with Diluted anti-PhtD Sera from Rabbits Immunized with Intact or Stressed monovalent PhtD Formulations in Passive Protection. In two studies, anti-PhtD sera obtained from rabbits immunized with intact or stressed PhtD monovalent formulations at 43-47° C. for 1 or 6 weeks, were tested in CBA/N mice in a passive protection model as described herein. Sera were diluted at the concentrations indicated (PBS was used as a control) and used to passively immunize CBA/N mice i.p. 50 cfu of A66.1^{Mn-} were delivered i.v 1 hour following passive immunization. Survival rates from both studies were combined and plotted.

FIG. 9. Representative Results from Concurrent SASSY & Passive Protection Studies. Concurrent SASSY and Passive Protection studies were carried out in three studies. Pooled anti-PcpA monoclonal antibodies (1-12, 1-29, 2B3, 6A4, and 9G11) were titrated starting at 20 μg/mL final concentration in a 1:2 dilution factor. Survival was plotted along with MFI signal from SASSY for each group. Representative results from one study are shown. A total of 5 different concentrations were tested.

FIG. 10. Plot depicting Correlation between SASSY and passive protection assay. Four independent preparations of A66.1 pre-incubated with antibodies were performed and a correlation between SASSY and passive protection was assessed using Spearman Coefficient. There was a very strong correlation between both the in vitro and in vivo assay with an R²=0.963.

FIG. 11. Use of in vitro competitive SASSY to assess the biological activity and stability of PcpA formulations. Degraded PcpA proteins were tested in both the competitive SASSY and passive protection in order to determine whether the competitive SASSY can be linked to a functional read out. Degraded proteins were first incubated with monoclonal antibodies at room temperature and A66.1 fresh culture was added to each sample to monitor both survival in mice and binding by detecting the MFI.

FIG. 12. Use of SASSY to detect repertoire expansion. To determine whether SASSY could be used to detect repertoire expansion, two monoclonal antibodies (4D5 and 8H6) were utilized alone and simultaneously to determine MIF. Duplicate points (series 1 and 2) are illustrated and are representative of three independent studies.

FIG. 13. SASSY using sera from human subject. Experiments were carried out to determine whether SASSY results would correlate with in vivo immunization studies in humans Sera from human subjects enrolled in a clinical trial and known to contain functional, PhtD-specific antibodies were tested in SASSY. A representative plot of subject #37 is shown where Bld3 represents post-vaccine sera and Bldl represents pre-vaccine serum.

FIG. 14. Identification of immunogenic PhtD peptides. Bleed 1 (Bld1) vs. bleen 3 (Bld3) sera from subject #37 were screened for binding to 15-mer overlapping peptides spanning PhtD and PhtE proteins using the ProArray peptide microarray (ProImmune). A positive signal was considered to have at least 10 K LU and only pairs with greater than 10 K LU are shown. Significant difference is considered to have at least a 3-fold increase (star) indicating a potentially new antibody epitope recognition in Bld3.

### SUMMARY OF THE DISCLOSURE

Disclosed herein are methods for characterizing drug products, the methods comprising contacting a drug product comprising one or more cell surface antigens with an antibody composition comprising antibodies reactive against at least one of the antigens to produce a test composition, contacting the test composition with a test cell (e.g., microorganism) expressing at least one of the cell surface antigens, and detecting the binding of antibodies to the test cell. In some embodiments, the results obtained by separately assaying two or more drug products may be compared to one another and/or to a control drug product to determine whether any such drug products are suitable for administration to a subject (e.g., human being). This disclosure also describes the surprising use of SASSY as a substitute for typical assay systems that measure vaccine immunogenicity and/or efficacy in human beings.

### DETAILED DESCRIPTION

The potency of a drug product containing antigens (e.g., a vaccine) typically depends upon the presence of immunogenic antigens therein (an “intact” drug product). It is therefore important to understand the quality of the antigens present in the drug product prior to administration to a host because potency of the drug product may be affected. As described herein, these problems are solved by an in vitro assay comprising the steps of contacting a drug product (e.g., test drug product) comprising one or more cell surface antigens with a composition comprising antibodies reactive against at least one of the antigens (e.g., antibody composition) to produce a test composition (e.g., potentially containing unbound antibodies), contacting the test composition with a test cell (e.g., microorganism, tumor cell, cell comprising a virus and thereby expressing one or more viral antigens, or a recombinant cell engineered to express one or more antigens) expressing at least one of the cell surface antigens; and, detecting the binding of any unbound antibodies against antigens in the drug product (e.g., in the test composition) that are bound to the test cell. In some embodiments, the assay may be carried out by including a “pre-incubation” step of contacting a drug product comprising antigens with an antibody composition comprising an amount of antibody sufficient to bind all or substantially all of the antigens in drug product where the drug product is in “intact” form. A drug product in “intact” form would typically contain an effective amount of drug (e.g., a suitable amount and form of antigen) to produce a therapeutic (or prophylactic) response in a host to which the drug product is administered. By contrast, a degraded (or, “stressed” as in the examples) drug product would contain less than an effective amount of drug (e.g., a less than suitable amount and form of antigen) to produce a therapeutic (or prophylactic) response in a host to which the drug product is administered. For example, an intact vaccine would typically produce a protective immune response while a degraded vaccine would not. Following incubation with an intact drug product (e.g., vaccine), there would be very little or no antibody (e.g., unbound antibody) available in the test composition for binding to cell surface antigen of the test cell (e.g., as all or substantially all of the antibody in the antibody composition is bound to antigens in the drug product). In contrast, where the drug product (e.g, vaccine) is degraded, all or substantially all of the antibodies in the antibody composition would not be bound to to the antigens in the drug product following pre-incubation (e.g., as some or all of the antigens in the drug product are degraded) and would be available for binding to the test cell. The binding of antibodies to the test cell may be measured by any suitable technique such as flow cytometry (e.g., where the test cell is in solution) or by another method where the test cell is affixed to a solid surface. It is noted that a test cell may be affixed to a solid support (e.g. a bead) and analyzed using flow cytometry. In such applications, the drug product / antibody complexes may be washed away such that the only antibody detected is that bound to the microorganism. This assay is generally referred to herein as the “competitive SASSY” system as it includes the competitive (e.g., pre-incubation of drug product with antibody composition) step. Typical SASSY systems do not include this competitive step as described in, for example, U.S. Pub. No. US 2012-0156211 A1 (U.S. Ser. No. 13/388,042) and U.S. Ser. No. 13/515,093 (WO 2011/075823 A1) (both of which being hereby incorporated by reference in their entirety into this application).

For example, competitive SASSY may be carried out as follows (e.g., where the test cell is a microorganism). An appropriate amount of drug product may be incubated with a composition comprising an appropriate amount of antibody (e.g., sera or monoclonal antibody diluted in, for instance, PBS, to the desired concentration) in an appropriate volume (e.g., 250 μL) (e.g., pre-incubation step). An appropriate amount of test microorganism (e.g., S. pneumoniae) may then be added to the drug product/antibody mixture. Incubation for an appropriate time (e.g., about any of 10, 20, 25, 30, 35, 40, 45, 50, 55 or 60 minutes) under suitable conditions (e.g., 37° C., 5% CO₂) may then be carried out, optionally followed by an appropriate washing step (e.g., washing two times in PBS). A secondary stain (e.g., Alexa-488 conjugated antibody against the species of sera used in the assay (e.g., goat anti-rabbit IgG or goat-anti-mouse-IgG) at a 1:100 dilution in PBS) may then be added followed by incubation under appropriate conditions (e.g., room temperature for 30 minutes). Another wash step may then be carried out to remove unbound antibody (e.g., wash twice with PBS), and the microrganism suspended in appropriate composition (e.g,. 0.5 ml, 1% paraformaldehyde). In some embodiments, the binding of antibodies to the microrganism may be determined using, for instance, flow cytometry (e.g., to determine mean fluorescent intensity (MFI)). The Examples describe a competitive SASSY using a representative control drug product (e.g., known “intact” (e.g, properly stored) PcpA or PhtD protein) and a representative test drug product (e.g., known “stressed” (e.g., heated) PcpA or PhtD protein), which were compared as surrogates for a control and test drug samples, respectively. Other methods may also be used, as would be apparent to one of ordinary skill in the art. For instance, in some embodiments, the microorganism could be affixed to a solid support (e.g., a bead, slide or plate). Methods for affixing microorganisms to such solid supports are well-known in the art. Methods for detecting such affixed microorganisms (e.g., antibodies attached thereto) are also well-known in the art.

The SASSY system (e.g., competitive SASSY) was also determined to be a suitable substitute for in vivo passive protection assays. A typical in vivo passive immunization system includes passive immunization of a source animal (e g , a rabbit) and administration of antisera generated in that animal to test animals (e.g., mice (e.g., CBA/CaHN-Btk^{xid}/J (abbreviated as CBA/N)). The antisera may be, for example, rabbit anti-sera diluted in PBS (e.g., 200 μl)) and may be administered to test animals via any suitable route (e.g., typically intraperitoneally (i.p.)). After an appropriate amount of time (e.g., one hour after administration of the antisera), the mice are typically challenged by a suitable route (typically intravenously (i.v.) with, e.g., 200 μl in the tail vain)) with a normally (e.g., in the unvaccinated state) lethal dose of a microorganism to which the antibodies may be reactive (e.g., S. pneumoniae strain A66.1 grown in manganese depleted THY/MOPS (A66.1^{Mn-}) at 50 cfu/mouse following passive adminstration of antibodies from a rabbit immunized by PcpA or PhtD protein). The examples demonstrate that the in vitro SASSY is a suitable substitute for the typical in vivo passive immunization model. In those studies, rabbits were immunized with “intact” antigen (e.g., properly stored PcpA or PhtD protein) or “stressed” antigen (e.g., heat-treated (45° C. for 1 week). The antisera obtained from the immunized rabbits (e.g., potentially containing anti-PcpA or PhtD antibodies) was pre-incubated with an appropriate amount of challenge microrganism (e.g., 1×10⁸ cfu of A66. 1^{Mn-}). The “intact” or “stressed” antisera was then either: 1) processed in a typical passive immunization assay; or, 2) processed using the competitive SASSY system (e.g., as described above). As described in the examples, the results of the typical passive immunization protocol and the SASSY system were comparable. Accordingly, then, the SASSY system may replace the step of administering the test antisera (e.g., rabbit antisera) to test animals (e.g., mice) (e.g., the typical in vivo passive immunization system); the user may obtain an indication of vaccine efficacy directly from the immunized animal sera (e.g., rabbit sera) in vitro using the SASSY system. Thus, in another embodiment, then, the problem of identifying protective vaccines is solved by a SASSY system comprising the steps of: administering a composition comprising an antigen to an animal (e g , a rabbit or human being), obtaining antibodies against the antigen from the animal (e g , as antisera), contacting the antibodies with a test cell (e.g., microorganism) expressing at least one of the cell surface antigens, and detecting the binding of antibodies to the microorganism. Typically, the final step may include a comparison of various test compositions (e.g., vaccines) to determine which induce a sufficient amount and/or type of antibodies in the animal such that protection against infection by an organism expressing the antigen may be provided thereby. As shown in the Examples, this assay system provides comparable results to an in vivo mouse model in which mice are challenged with a microorganism with or without pre-treatment with antibodies produced by another animal (e.g., a rabbit). The competitive SASSY systems described herein provide the researcher with alternatives to in vivo passive protection assays.

The SASSY systems described herein provides the researcher with alternatives to actual clinical trials that may quickly ascertain whether a potential vaccine would be protective in humans While the typical SASSY system (e.g., not including the competitive step) has been used with monoclonal antibodies, for instance, it was surprising to find that it could be used with sera isolated from vaccinated human beings. As described in the examples, sera from human beings enrolled in a clinical trial and known to contain functional, vaccine-specific antibodies (e.g., containing PhtD antigens) by standard assays were tested using the SASSY system. Post-vaccine sera saturated at a higher MFI than the pre-vaccine serum when sera sample pairs were tested. Thus, SASSY may be substituted for typical assay systems that have been used to measure vaccine efficacy in human beings. This is surprising given the vastly different nature of a monoclonal antibody preparation and the much lesser amount of antibody to a particular antigen (e.g., PhtD) typically present in the serum of a vaccinated human being. Given these surprising results using human serum, it should be understood by those of ordinary skill in the art that SASSY would also be useful for detecting immune responses in organisms other than humans (e.g., other mammals)

Drug products may include, for example, immunological compositions. An immunological composition is one that, upon administration to a host (e.g., human) induces or enhances an immune response directed against one or more antigens and/or immunogens contained within the composition. The types of antigens contained in such immunological compositions may vary. For example, one or more of the antigens may be a protein, peptide, carbohydrate, lipid, or small molecule Immunological compositions may also include one or more adjuvants. The immune response may include the generation of antibodies (e.g, through the stimulation of B cells) and/or a T cell-based response (e.g., a cytolytic response). The SASSY systems described herein typically but not necessarily relates to drug products that induce the production of antibodies (e.g., an antibody-based immune response) upon administration to a host. The immune responses may or may not be protective or neutralizing A protective or neutralizing immune response is one that may be detrimental to the cell containing or expressing the antigen (e.g., from which the antigen was derived) by inhibiting the growth and/or eliminating the same from a host and therefore benefit the host (e.g., by reducing or preventing infection and/or tumor growth). As used herein, protective or neutralizing antibodies are typically reactive to antigens thereof. An immunological composition that, upon administration to a host, results in a protective or neutralizing immune response may be considered a vaccine.

The term “antibody” or “antibodies” may refer to whole or fragmented antibodies in unpurified or partially purified form (e.g., hybridoma supernatant, ascites, polyclonal antisera, serum) and/or in purified form, and/or to derivatives of antibodies. A purified antibody may be one that is separated from at least about 50%, 55%, 60%, 65%, 70%, 75%, 80%, 85%, 90%, or 95% of the proteins with which it is initially found (e.g., as part of a hybridoma supernatant or ascites preparation). The antibodies may be of any suitable origin or form including, for example, murine (e.g., produced by murine hybridoma cells), or expressed as humanized antibodies, chimeric antibodies, human antibodies, and the like. For instance, antibodies may be of any suitable type including, for example, human (e.g., IgG (IgG1, IgG2, IgG3, IgG4), IgM, IgA (IgA1 and IgA2), IgD, and IgE), canine (e.g., IgGA, IgGB, IgGC, IgGD), chicken (e.g., IgA, IgD, IgE, IgG, IgM, IgY), goat (e.g., IgG), mouse (e.g., IgG, IgD, IgE, IgG, IgM), pig (e.g., IgG, IgD, IgE, IgG, IgM), rat (e.g., IgG, IgD, IgE, IgG, IgM) and/or a fragment and/or derivative thereof (e.g., as chimeric antibodies). Suitable derivatives may include, for example, an Fab, F(ab′)₂, Fab′ single chain antibody, Fv, single domain antibody, mono-specific antibody, bi-specific antibody, tri-specific antibody, multi-valent antibody, chimeric antibody, canine-human chimeric antibody, canine-mouse chimeric antibody, antibody comprising a canine Fc, humanized antibody, human antibody, caninized, CDR-grafted antibody, shark antibody, nanobody (e.g., antibody consisting of a single monomeric variable domain), camelid antibody (e.g., antibodies of members of the Camelidae family), microbody, intrabody (e.g., intracellular antibody), or mimetic. Mimetics may als include, for example, organic compounds that specifically bind CHV-like virus or an antigen thereof such as, for example, an affibody (Nygren, et al., FEBS J. 275(11):2668-76, 2008), affilin (Ebersbach, et al., J. Mol. Biol. 372 (1):172-85, 2007), affitin (Krehenbrink et al., J. Mol. Biol. 383(5):1058-68, 2008), anticalin (Skerra, A., FEBS J. 275(11):2677-83, 2008), avimer (Silverman et al., Nat. Biotechnol. 23(12): 1556-61, 2005), DARPin (Stumpp et al., Drug Discov. Today 13(15-16):695-701, 2008), Fynomer (Grabulovski et al., J. Biol. Chem. 282(5):3196-3204, 2007), Kunitz domain peptide (Nixon et al., Curr. Opin. Drug Discov. Devel. 9(2):261-8, 2006), and/or a monobody (Koide et al., Methods Mol. Biol. 352:95-109, 2007). Other antibodies may also be suitable as would be understood by one of ordinary skill in the art.

Methods of preparing and utilizing various types of antibodies (e.g, as antibody compositions) are well-known to those of skill in the art and would be suitable in practicing the present invention (see, for example, Harlow, et al. Antibodies: A Laboratory Manual, Cold Spring Harbor Laboratory, 1988; Harlow, et al., Using Antibodies: A Laboratory Manual, Portable Protocol No. 1, 1998; Kohler and Milstein, Nature, 256:495, 1975; Jones et al., Nature, 321:522-525, 1986; Riechmann et al., Nature, 332:323-329, 1988; Presta, Curr. Op. Struct. Biol., 2:593-596, 1992; Verhoeyen et al., Science, 239:1534-1536, 1988; Hoogenboom et al., J. Mol. Biol., 227:381, 1991; Marks et al., J. Mol. Biol., 222:581, 1991; Cole et al., Monoclonal Antibodies and Cancer Therapy, Alan R Liss, p. 77, 1985; Boerner et al., J. Immunol., 147(1):86-95, 1991; Marks et al., Bio/Technology 10, 779-783, 1992; Lonberg et al., Nature 368:856-859, 1994; Morrison, Nature 368:812-13, 1994; Fishwild et al., Nature Biotechnology 14, 845-51, 1996; Neuberger, Nature Biotechnology 14, 826, 1996; Lonberg and Huszar, Intern. Rev. Immunol. 13:65-93, 1995; as well as U.S. Pat. Nos. 4,816,567, 5,545,807, 5,545,806, 5,569,825, 5,625,126, 5,633,425, and 5,661,016). Serum may be isolated from a host to which the drug product has been administered using standard techniques, for example Typically, serum is isolated from the host at least about seven, 14 or 21 days after administration of the drug product. Serum may also be isolated at various timepoints and the appropriate timepoint for use in the assays described herein selected. In certain applications, the antibodies may be contained within hybridoma supernatant or ascites and utilized either directly as such or following concentration using standard techniques. In other applications, the antibodies may be further purified using, for example, salt fractionation and ion exchange chromatography, or affinity chromatography using Protein A, Protein G, Protein A/G, and/or Protein L ligands covalently coupled to a solid support such as agarose beads, or combinations of these techniques. The antibodies may be stored in any suitable format, including as a frozen preparation (e.g., −20° C. or −70° C.), in lyophilized form, or under normal refrigeration conditions (e.g., 4° C.). When stored in liquid form, a suitable buffer such as Tris-buffered saline (TBS) or phosphate buffered saline (PBS) may be utilized. The amount of antibody in the antibody compositions may vary and be selected by the user depending on the particular SASSY system being used. As described above, in some embodiments, the drug product is pre-incubated with an antibody composition comprising a sufficient amount of antibody to bind all (or most) of the antigens present therein (e.g., about any of 0.1, 0.2, 0.3, 0.4, 0.5, 0.6, 0.7, 0.8, 0.9, 1, 2, 3, 4, 5, 6, 7, 8, 9, or 10 μg/ml). This would necessarily vary depending on the type of drug product (e.g., the nature and/or amount of the antigens contained therein).

In certain embodiments, detection of the antibody may be accomplished by contacting the antigen-antibody complex with a “second” antibody that is immunologically reactive with immunoglobulin (e.g., anti-immunoglobulin antibody) for a time and under conditions sufficient for the second antibody to bind to the immunoglobulin in the complex and then detecting the bound second antibody. It is preferred that the second antibody is labelled with a detectable label, marker or reporter molecule. Suitable labels, markers, and/or reporter molecules may include, for example, fluorochromes such as fluorescein, rhodamine, phycoerythrin, Europium and Texas Red; chromogenic dyes such as diaminobenzidine, radioisotopes; macromolecular colloidal particles or particulate material such as latex beads that are coloured, magnetic or paramagnetic; binding agents such as biotin and digoxigenin; and, biologically or chemically active agents that can directly or indirectly cause detectable signals to be visually observed, electronically detected or otherwise recorded, for example in a FACS, ELISA, western blot, TRFIA, immunohistochemistry, evanescence, Luminex bead array, dipstick, or other lateral flow assay format. Suitable antibody-binding molecules for use in such methods may include immunoglobulin-binding antibodies, for example anti-human antibodies (e.g., anti-human antibodies specific for Ig isotypes or subclasses (e.g., of IgG), or specific for Staphylococcal protein A or G. Other reagents for detecting antibodies may also be suitable, as would be understood by one of ordinary skill in the art.

In some embodiments, detection of antibodies on the test cell is accomplished using a detection system which requires measur the mean fluorscence (e.g., mean flourescence indicator) (“MFI”)). Flow cytometry is one such detection system. The MFI serves as an indicator of the amount of antibody on the test cell surface (e.g., a higher MFI indicates a greater amount of antibody detected). In some embodiments, a protective vaccine may be identified by measuring MFI (e.g., as described in the examples, an MFI of 111 or higher may indicate a vaccine may be protective). The relevant MFI would depend upon a particular drug product. Two drug products assayed by competitive SASSY may exhibit different MFIs where the antigen content differs between the two. For instance, where a first drug product (e.g., an intact drug product) exhibited a competitive SASSY MFI of 100 and a second drug product exhibited a competitive SASSY MFI of 200, one may conclude that the second drug product is not intact (or less intact) relative to the first drug product. Where the second drug product exhibits a competitive SASSY MFI similar (e.g., ±10%) to that of the first drug product, it may be concluded that both the first and second drug products are intact (e.g., at least relative to one another). In some embodiments, the difference in MFI between two products may be considered, at least generally, significant (e.g., a difference of about any of 15%, 20%, 25%, 30%, 35%, 40%, or higher) depending on the particular application and/or drug product. The first and second drug products may, in some embodiments, represent different lots of the same vaccine. As such, competitive SASSY may provide a potency assay that may be used to confirm lot-to-lot stability. Other detection systems may also depend upon measurement of MFI as would be understood by those of ordinary skill in the art. Other MFI levels may also be relevant and/or applicable to particular methods, as would also be understood by those of ordinary skill in the art.

The test cell may be, for example, any cell expressing at least one antigen of interest such as a microorganism, tumor cell, cell comprising a virus (e.g., and thereby expressing one or more viral antigens) and/or virus-like particle, and/or a recombinant cell engineered to express one or more antigens of interest. The test cell may be free in solution. The test cell may also be adjoined to a capture or detection reagent and/or affixed (e.g., immobilized) on a solid support such as a bead (e.g., a magnetic bead), tube, microplate well, chip, and/or column material. Exemplary microrganisms include, for example, any one or more bacterial species (spp.) (e.g., bacterial target antigen(s)) including, for example, Bacillus spp. (e.g., Bacillus anthracis), Bordetella spp. (e.g., Bordetella pertussis), Borrelia spp. (e.g., Borrelia burgdorferi), Brucella spp. (e.g., Brucella abortus, Brucella canis, Brucella melitensis, Brucella suis), Campylobacter spp. (e.g., Campylobacter jejuni), Chlamydia spp. (e.g., Chlamydia pneumoniae, Chlamydia psittaci, Chlamydia trachomatis), Clostridium spp. (e.g., Clostridium botulinum, Clostridium difficile, Clostridium perfringens, Clostridium tetani), Corynebacterium spp. (e.g., Corynebacterium diptheriae), Enterococcus spp. (e.g., Enterococcus faecalis, enterococcus faecum), Escherichia spp. (e.g., Escherichia coli), Francisella spp. (e.g., Francisella tularensis), Haemophilus spp. (e.g., Haemophilus influenza), Helicobacter spp. (e.g., Helicobacter pylori), Legionella spp. (e.g., Legionella pneumophila), Leptospira spp. (e.g., Leptospira interrogans), Listeria spp. (e.g., Listeria monocytogenes), Mycobacterium spp. (e.g., Mycobacterium leprae, Mycobacterium tuberculosis), Mycoplasma spp. (e.g., Mycoplasma pneumoniae), Neisseria spp. (e.g., Neisseria gonorrhea, Neisseria meningitidis), Porphyromonas spp. (e.g., P. Gingavalis), Pseudomonas spp. (e.g., Pseudomonas aeruginosa), Rickettsia spp. (e.g., Rickettsia rickettsii), Salmonella spp. (e.g., Salmonella typhi, Salmonella typhinurium), Shigella spp. (e.g., Shigella sonnei), Staphylococcus spp. (e.g., Staphylococcus aureus, Staphylococcus epidermidis, Staphylococcus saprophyticus, coagulase negative staphylococcus (e.g., U.S. Pat. No. 7,473,762)), Streptococcus spp. (e.g., Streptococcus agalactiae, Streptococcus pneumoniae, Streptococcus pyrogenes), Treponema spp. (e.g., Treponema pallidum), Vibrio spp. (e.g., Vibrio cholerae), and Yersinia spp. (Yersinia pestis). Additional microorganisms may include, for example, one or more parasitic organisms (spp.) (e.g., parasite target antigen(s)) including, for example, Ancylostoma spp. (e.g., A. duodenale), Anisakis spp., Ascaris lumbricoides, Balantidium coli, Cestoda spp., Cimicidae spp., Clonorchis sinensis, Dicrocoelium dendriticum, Dicrocoelium hospes, Diphyllobothrium latum, Dracunculus spp., Echinococcus spp. (e.g., E. granulosus, E. multilocularis), Entamoeba histolytica, Enterobius vermicularis, Fasciola spp. (e.g., F. hepatica, F. magna, F. gigantica, F. jacksoni), Fasciolopsis buski, Giardia spp. (Giardia lamblia), Gnathostoma spp., Hymenolepis spp. (e.g., H. nana, H. diminuta), Leishmania spp., Loa loa, Metorchis spp. (M. conjunctus, M. albidus), Necator americanus, Oestroidea spp. (e.g., botfly), Onchocercidae spp., Opisthorchis spp. (e.g., O. viverrini, O. felineus, O. guayaquilensis, and O. noverca), Plasmodium spp. (e.g., P. falciparum), Protofasciola robusta, Parafasciolopsis fasciomorphae, Paragonimus westermani, Schistosoma spp. (e.g., S. mansoni, S. japonicum, S. mekongi, S. haematobium), Spirometra erinaceieuropaei, Strongyloides stercoralis, Taenia spp. (e.g., T. saginata, T. solium), Toxocara spp. (e.g., T. canis, T. cati), Toxoplasma spp. (e.g., T. gondii), Trichobilharzia regenti, Trichinella spiralis, Trichuris trichiura, Trombiculidae spp., Trypanosoma spp., Tunga penetrans, and/or Wuchereria bancrofti. Other types of test microorganisms may also be suitable, as would be understood by one of ordinary skill in the art.

The test cell may also be or be derived from or relate to a tumor cell. Exemplary tumor types from which such cells may be derived include, for instance, breast, colon, lung, stomach, sarcoma, blood cancer (e.g., leukemia), cervix, ovary, testicle, brain, kidney, liver, throat, skin (e.g, melanoma), pancreas, and the like. The antigens of interest of such tumor cells may be, for instance, cancer-testis (CT) antigens (i.e., MAGE, NY-ESO-1); melanocyte differentiation antigens (i.e., Melan A/MART-1, tyrosinase, gp100); mutational antigens (i.e., MUM-1, p53, CDK-4); overexpressed ‘self’ antigens (i.e., HER-2/neu, p53); viral antigens (i.e., HPV, EBV) (e.g., gp100 (Cox et al., Science, 264:716-719 (1994)), MART-1/Melan A (Kawakami et al., J. Exp. Med., 180:347-352 (1994)), gp75 (TRP-1) (Wang et al., J. Exp. Med., 186:1131-1140 (1996)), tyrosinase (Wolfel et al., Eur. J. Immunol., 24:759-764 (1994)), NY-ESO-1 (WO 98/14464; WO 99/18206), melanoma proteoglycan (Hellstrom et al., J. Immunol., 130:1467-1472 (1983)), MAGE family antigens (i.e., MAGE-1, 2,3,4,6, and 12; Van der Bruggen et al., Science, 254:1643-1647 (1991); U.S. Pat. Nos. 6,235,525), BAGE family antigens (Boel et al., Immunity, 2:167-175 (1995)), GAGE family antigens (i.e., GAGE-1,2; Van den Eynde et al., J. Exp. Med., 182:689-698 (1995); U.S. Pat. No. 6,013,765), RAGE family antigens (i.e., RAGE-1; Gaugler et at., Immunogenetics, 44:323-330 (1996); U.S. Pat. No. 5,939,526), N-acetylglucosaminyltransferase-V (Guilloux et at., J. Exp. Med., 183:1173-1183 (1996)), p15 (Robbins et al., J. Immunol. 154:5944-5950 (1995)), β-catenin (Robbins et al., J. Exp. Med., 183:1185-1192 (1996)), MUM-1 (Coulie et al., Proc. Natl. Acad. Sci. USA, 92:7976-7980 (1995)), cyclin dependent kinase-4 (CDK4) (Wolfel et al., Science, 269:1281-1284 (1995)), p21-ras (Fossum et at., Int. J. Cancer, 56:40-45 (1994)), BCR-abl (Bocchia et al., Blood, 85:2680-2684 (1995)), p53 (Theobald et al., Proc. Natl. Acad. Sci. USA, 92:11993-11997 (1995)), p185 HER2/neu (erb-B1; Fisk et al., J. Exp. Med., 181:2109-2117 (1995)), epidermal growth factor receptor (EGFR) (Harris et al., Breast Cancer Res. Treat, 29:1-2 (1994)), carcinoembryonic antigens (CEA) (Kwong et al., J. Natl. Cancer Inst., 85:982-990 (1995) U.S. Pat. Nos. 5,756,103; 5,274,087; 5,571,710; 6,071,716; 5,698,530; 6,045,802; EP 263933; EP 346710; and, EP 784483); carcinoma-associated mutated mucins (i.e., MUC-1 gene products; Jerome et al., J. Immunol., 151:1654-1662 (1993)); EBNA gene products of EBV (i.e., EBNA-1; Rickinson et al., Cancer Surveys, 13:53-80 (1992)); E7, E6 proteins of human papillomavirus (Ressing et al., J. Immunol, 154:5934-5943 (1995)); prostate specific antigen (PSA; Xue et al., The Prostate, 30:73-78 (1997)); prostate specific membrane antigen (PSMA; Israeli, et al., Cancer Res., 54:1807-1811 (1994)); idiotypic epitopes or antigens, for example, immunoglobulin idiotypes or T cell receptor idiotypes (Chen et al., J. Immunol., 153:4775-4787 (1994)); KSA (U.S. Pat. No. 5,348,887), kinesin 2 (Dietz, et al. Biochem Biophys Res Commun 2000 Sep. 7;275(3):731-8), HIP-55, TGFβ-1 anti-apoptotic factor (Toomey, et al. Br J Biomed Sci 2001;58(3):177-83), tumor protein D52 (Bryne J. A., et al., Genomics, 35:523-532 (1996)), H1FT, NY-BR-1 (WO 01/47959), NY-BR-62, NY-BR-75, NY-BR-85, NY-BR-87 and NY-BR-96 (Scanlan, M. Serologic and Bioinformatic

Approaches to the Identification of Human Tumor Antigens, in Cancer Vaccines 2000, Cancer Research Institute, New York, N.Y.), and/or pancreatic cancer antigens (e.g., SEQ ID NOS: 1-288 of U.S. Pat. No. 7,473,531)). Other test cells expressing these or other tumor antigens may also be suitable, as would be understood by one of ordinary skill in the art.

The test cell may also comprise and/or express viral antigens. Exemplary viruses may include, for instance, one or more viruses (e.g., viral target antigen(s)) including, for example, a dsDNA virus (e.g. adenovirus, herpesvirus, epstein-barr virus, herpes simplex type 1, herpes simplex type 2, human herpes virus simplex type 8, human cytomegalovirus, varicella-zoster virus, poxvirus); ssDNA virus (e.g., parvovirus, papillomavirus (e.g., E1, E2, E3, E4, E5, E6, E7, E8, BPV1, BPV2, BPV3, BPV4, BPV5 and BPV6 (In Papillomavirus and Human Cancer, edited by H. Pfister (CRC Press, Inc. 1990); Lancaster et al., Cancer Metast. Rev. pp. 6653-6664 (1987); Pfister, et al. Adv. Cancer Res 48, 113-147 (1987)); dsRNA viruses (e.g., reovirus); (+)ssRNA viruses (e.g., picornavirus, coxsackie virus, hepatitis A virus, poliovirus, togavirus, rubella virus, flavivirus, hepatitis C virus, yellow fever virus, dengue virus, west Nile virus); (−)ssRNA viruses (e.g., orthomyxovirus, influenza virus, rhabdovirus, paramyxovirus, measles virus, mumps virus, parainfluenza virus, respiratory syncytial virus, rhabdovirus, rabies virus); ssRNA-RT viruses (e.g. retrovirus, human immunodeficiency virus (HIV) (e.g., any of subtypes A1, A2, A3, A4, B, C, D, E, F1, F2, G, H, J and K)); and, dsDNA-RT viruses (e.g. hepadnavirus, hepatitis B). Other test cells expressing these or other viral antigens may also be suitable, as would be understood by one of ordinary skill in the art.

A test cell may also be one engineered to transiently or stably express one or more antigens derived from, for instance, a microorganism, tumor cell, and/or virus. Such cells may be animal (e g , mammalian) or non-mammalian Exemplary cells types suitable for use in producing such recombinant test cells may include, for instance, 293, BHK, CHO (e.g., CHO-T Ag; U.S. Pat. No. 5,122,469), CV-1, COS (e.g., CosV7), HBMF, HeLa, HuH7, human keratinocytes, K562, MG63, P338D1, PC-12, Raw264.7, SK-N-MC, THP-1, U937, W12, WI38, and the like. Methods for producing recombinant cells are widely available to those of ordinary skill in the art. For instance, a nucleic acid molecule encoding one or more antigens may be transfected or otherwise transferred to a cell and the antigen expressed therein. For use with the SASSY systems as described herein, the antigen would typically be expressed on the cell surface. Methods for recombinantly expressing antigens (e.g., any of those described above) on the cell surface are widely available to those of ordinary skill in the art (e.g., as described in U.S. Pat. Nos. 5,665,590; 6,686,168; and/or 7,125,973). Other suitable recombinant test cells may also be suitable, as would be understood by one of ordinary skill in the art.

The reagents described herein may be provided in kit format. A kit may include, for instance, some or all of the components necessary to carry out the assays described herein. For instance, the kit may comprise control compositions (e.g., control drug product and/or control antibody compositions), test cells (e.g., as free cells, affixed to a solid support, and/or frozen), buffers, labeling reagents (e.g., labeled antibodies such as goat anti-mouse IgG biotin, streptavidin-HRP conjugates, allophycocyanin, B-phycoerythrin, R-phycoerythrin, peroxidase, and/or other detectable labels), instructions and any other necessary or useful components. The components of the kit may be provided in any suitable form, including frozen, lyophilized, or in a pharmaceutically acceptable buffer such as TBS or PBS. The kit may also include a solid support containing one or more test cells (e.g., microorganisms) in any suitable form. The kits may also include other reagents and/or instructions for carrying out assays such as, for example, flow cytometric analysis, ELISA, immunoblotting (e.g., western blot), in situ detection, immunocytochemistry, immunhistochemistry, and/or visualization of data. Kits may also include components such as containers (e.g., tubes) and/or slides pre-formatted to containing control samples and/or reagents with additional space (e.g., tubes, slides and/or space on a slide) for experimental samples. The kit may also comprise one or both of an apparatus for handling and/or storing the sample obtained from the individual and an apparatus for obtaining the sample from the individual (i.e., a needle, lancet, and collection tube or vessel). Other embodiments are also provided as would be understood by one of ordinary skill in the art.

Thus, this disclosure describes, inter alia, methods comprising contacting a drug product comprising one or more antigens with an antibody composition comprising antibodies reactive against at least one of the one or more antigens to produce a test composition, contacting the test composition with a test cell expressing at least one of the antigens, and detecting the binding of antibodies to the test cell. In certain embodiments, the antigen is a cell surface antigen; the test cell is selected from the group consisting of a microorganism, tumor cell, cell expressing a viral antigen, or a recombinant cell; the antibody composition is selected from the group consisting of serum, ascites, cell culture supernatant, polyclonal antisera, a monoclonal antibody composition, and mixtures thereof; the serum is derived from an animal immunized with an antigen present in the drug product; the test cell is in solution or affixed to a solid surface; detection of antibodies on the test cell is by flow cytometry; detection of antibodies on the test cell indicates the drug product is not intact; and/or a lack of detection of antibodies on the microorganism indicates the drug product is intact. In some embodiments, the methods may comprising separately carrying out the steps using first and second drug products and comparing the amount of antibody detected on the test cell for the methods carried out using first and second drug products, respectively. In some embodiments, the first drug product is a control drug product. In certain embodiments, the detection of more antibody following incubation with the second drug product as compared to the first drug product indicates that second drug product is less intact or not intact relative to the first drug product (e.g., where the first and second drug product are different lots of the same drug product). The method may provide, for example, a drug product potency assay. In certain embodiments, the drug product may be an immunological composition and/or vaccine. Kits comprising components required for carrying out a control reaction and instructions for use are also provided. This disclosure also describes the surprising use of SASSY as a substitute for typical assay systems that measure vaccine immunogenicity and /or efficacy in mammals (e.g., human beings). For instance, the disclosure describes methods for determining vaccine efficacy in a mammal comprising contacting mammalian sera with a test cell expressing at least one cell surface antigen with which the mammal was previously vaccinated; and, detecting the binding of antibodies of the sera, if present therein, to the test cell. In some embodiments, an additional step of comparing the binding of antibodies of a first sera sample of a mammal to whom the vaccine was not previously administered to the binding of antibodies from a second sera sample of the mammal following administration of the vaccine (e.g., seven or more days following administration) is included in the method.

Any indication that a feature is optional is intended provide adequate support for claims that include closed or exclusive or negative language with reference to the optional feature. Exclusive language specifically excludes the particular recited feature from including any additional subject matter. For example, if it is indicated that A can be drug X, such language is intended to provide support for a claim that explicitly specifies that A consists of X alone, or that A does not include any other drugs besides X. “Negative” language explicitly excludes the optional feature itself from the scope of the claims. For example, if it is indicated that element A can include X, such language is intended to provide support for a claim that explicitly specifies that A does not include X. Non-limiting examples of exclusive or negative terms include “only,” “solely,” “consisting of,” “consisting essentially of,” “alone,” “without”, “in the absence of (e.g., other items of the same type, structure and/or function)” “excluding,” not including“, “not”, “cannot,” or any combination and/or variation of such language.

All publications and patents cited in this specification are herein incorporated by reference in their entirety as if each individual publication or patent were specifically and individually indicated to be incorporated by reference. Genbank records referenced by GID or accession number, particularly any polypeptide sequence, polynucleotide sequences or annotation thereof, are incorporated by reference herein. The citation of any publication is for its disclosure prior to the filing date and should not be construed as an admission that the present invention is not entitled to antedate such publication by virtue of prior invention.

Certain embodiments are further described in the following examples. These embodiments are provided as examples only and are not intended to limit the scope of the claims in any way.

### EXAMPLES

### Example 1

### Materials and Methods

The protein content and the antigenicity of samples either stored at 2-8° C. or stressed at 43-47° C. for 1, 4, or 6 weeks were tested by HPLC, ELISA and Biacore. The HPLC results indicated that monovalent PcpA protein content dropped by 20% following storage at 43-47° C. for 1 week and 40% following 4 weeks storage as compared to time 0. For both the ELISA and Biacore, two tests were performed upon the receipt of the samples and 5 months later to monitor their stability at 2-8° C. following the initial accelerated degradation procedure. Both tests performed within a 5 month period have shown that the protein stored at 2-8° C. does not undergo further degradation. ELISA, using protective monoclonal antibody 2B3, was able to detect a 50% drop in antigenicity following 1 week of storage and an 80-85% drop following either 4 or 6 weeks storage at 43-47° C. In contrast, Biacore (which utilizes polyclonal sera) indicates a 27% drop in antigenicity at 1 week and 43-47% drop at 4 and 6 week storage. These results indicate that monoclonal antibodies are more sensitive to protein degradation than polyclonal sera. Although using a monoclonal antibody may be a risk because it may be sensitive to degradations occurring only to one region of the protein and may not detect other possible degradation patterns, it is still relevant to use this reagent as it indicates whether this epitope which is known to confer protection is still intact. Table 1, illustrates those results.

TABLE 1
Determination of PcpA Content in Intact and Stressed Formulations

| | Monovalent PcpA Protein | | | | |
|---|---|---|---|---|---|
| | Target concentration 100 μg/mL | | | | |
| | | μg/mL by ELISA | | μg/mL by | |
| | μg/mL | (mAb 2B3) | | Biacore (pAb) | |
| Time | by HPLC | | Time 0 + | | Time 0 + |
| (43-47° C.) | Time 0 | Time 0 | 5 months | Time 0 | 5 months |
| 0 | 94.2 | 128.4 | 112.1 | 112.1 | 106.7 |
| 1 W | 74.1 | 61.7 | 53.9 | 81 | 89.4 |
| 4 W | 55.6 | 25.5 | 24.2 | 63 | 63.2 |
| 6 W | 48.9 | 17.3 | 20.1 | 59.4 | 57.7 |

In term of PhtD, while the HPLC data indicated a gradual drop in protein content (27 and 35% drop at 1 week and 4 weeks respectively), the Biacore data did not show any significant drop in antigenicity. On the other hand, the ELISA data using a non protective monoclonal antibody 9E11 was able to detect a 30% and 60% drop in antigenicity at 1 week and 4 weeks storage at 43-47° C., respectively. The results shown in Table 2 indicate that polyclonal sera may be masking the loss in antigenicity due to antibodies binding to different regions of the protein that may not be compromised by the accelerated degradation.

TABLE 2
Determination of PhtD Content in Intact and Stressed Formulations

| | Monovalent PhtD Protein | | | | |
|---|---|---|---|---|---|
| | Target concentration 100 μg/mL | | | | |
| | | μg/mL by ELISA | | μg/mL by | |
| | | (mAb 9E11) | | Biacore (pAb) | |
| Time | μg/mL by HPLC | | Time 0 + | Time | Time 0 + |
| (43-47° C.) | Time 0 | Time 0 | 5 months | 0 | 5 months |
| 0 | 87.2 | 94.8 | 94.9 | 80.4 | 86.1 |
| 1 W | 62.7 | 56.8 | 63.4 | 73.9 | 77 |
| 4 W | 56 | ND | 31.0 | 74.3 | 80.9 |
| 6 W | 51.8 | ND | ND | 77 | 87 |

As explained, these analytical results were compared to potency assessment performed using competitive SASSY.

### Example 2

### In Vitro Competitive SASSY

A. Competitive SASSY using anti-PcpA Polyclonal Sera or Monoclonal Antibodies to Test Intact or Stressed Formulations

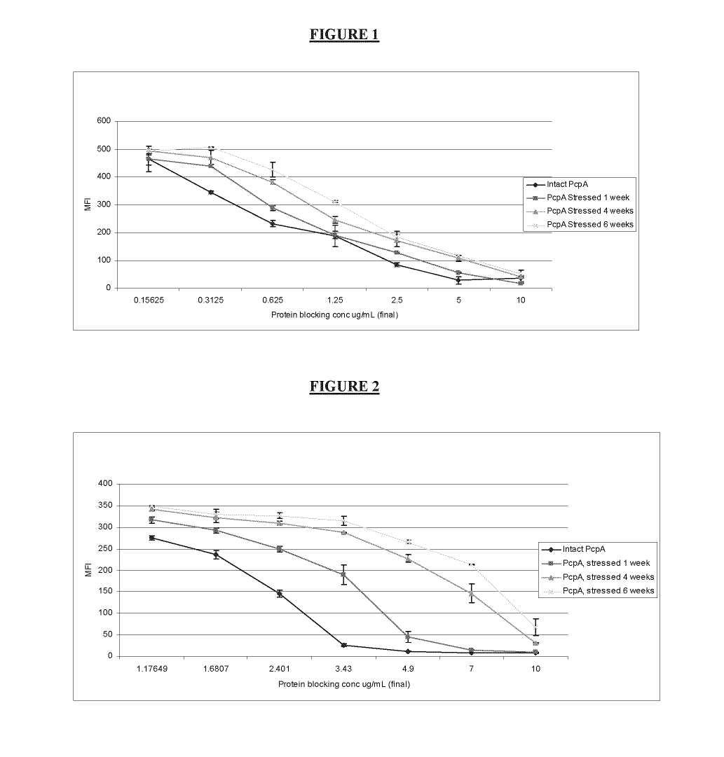

The protein formulations as described above were used in a competitive SASSY with either polyclonal sera or monoclonal antibodies that include 2B3. FIG. 1 represents intact or degraded PcpA competing with A66.1 bacterial strain for binding to polyclonal sera. As expected intact PcpA protein was able to bind the polyclonal sera with lower amount of protein compared to protein stored at 1 or 4 or 6 weeks indicating that all the antigenic PcpA epitopes are accessible and intact in the pre-degradation step. On the other hand, through degradation of the protein, the epitopes bind less efficiently to the polyclonal sera and as a consequence a higher binding to the bacteria is detected. In order to measure their relative potency to intact protein, the area under the curve for each test sample was calculated and the inverse ratio to the intact protein is shown in Table 3. The potency of the 1 week stressed sample is 0.8 and 0.4-0.5 for 4 and 6 weeks relative to intact protein. Interestingly, these results are very similar to the Biacore data which utilized polyclonal sera as well.

TABLE 3
PcpA Relative Potency - Polyclonal sera

| | Relative | Relative | Relative | Relative |
|---|---|---|---|---|
| | potency | potency | potency | potency |
| Date | 0 wk | 1 wk | 4 wk | 6 wk |
| Exp1 | 1 | 0.70 | 0.48 | 0.44 |
| Exp2 | 1 | 0.90 | 0.61 | 0.52 |
| Average | 1 | 0.80 | 0.55 | 0.48 |

Degraded PcpA samples were also assessed in the competitive SASSY using a pool of five monoclonal antibodies that bind to different regions on the proteins. Among those antibodies, 2B3 was also used in the antigenic ELISA and both 2B3 and 1-12 were shown to be protective in the passive protection model when combined together. Interestingly, the relative potency of PcpA stored for 1 week was 0.51 and 0.25, 0.21 for 4 and 6 week storage at 43-47° C., respectively. (FIG. 2 and Table 4). These results contrast with previous results obtained with polyclonal sera and are in alignment with the antigenicity data using the same monoclonal antibody 2B3, indicating that the competitive SASSY is stability indicating and reflects the antigenicity results obtained with the same monoclonal antibody. Furthermore, the differences observed between the Biacore data and ELISA data are likely due to the reagents used rather than the technology.

TABLE 4
PcpA Relative Potency-monoclonal antibodies

| | Relative | | | |
|---|---|---|---|---|
| | potency | Relative | Relative | Relative |
| Date | 0 wk | potency 1 wk | potency 4 wk | potency 6 wk |
| exp1 | 1 | 0.53 | 0.25 | 0.21 |
| exp2 | 1 | 0.49 | 0.24 | 0.20 |
| average | 1 | 0.51 | 0.25 | 0.21 |

B. Competitive SASSY with anti-PhtD Monoclonal Antibodies or Polyclonal Sera with Intact or Stressed Formulations

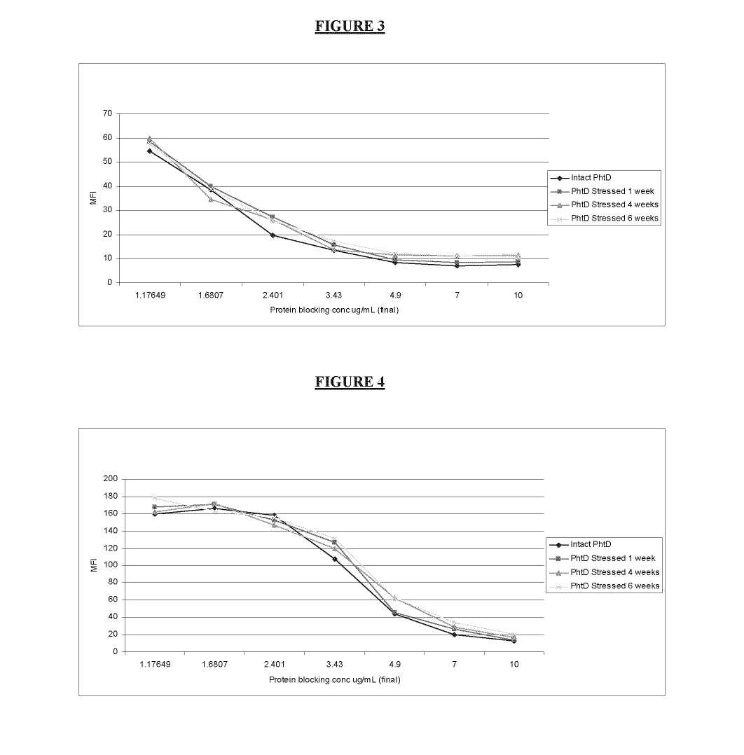

PhtD formulations stored at 43-47° C. were also tested by competitive SASSY using either polyclonal sera specific for PhtD or monoclonal antibodies that bind to different regions of the protein. Among those monoclonal antibodies tested 4D5 and 8H6 were shown to protect mice against WU2 strain when injected in combination. Results as illustrated in FIG. 3 using polyclonal sera do not indicate a reduction in potency of samples stored for either 1, 4 or 6 weeks relative to intact PhtD stored at 2-8° C. These results are very similar to Biacore results which also used polyclonal sera.

TABLE 5
PhtD Relative Potency - Polyclonal

| Relative | Relative potency | Relative | Relative |
|---|---|---|---|
| potency 0 wk | 1 wk | potency 4 wk | potency 6 wk |
| 1 | 0.86 | 0.81 | 0.77 |

The potency of degraded samples was also determined in the competitive SASSY using monoclonal antibodies. Results have shown that the relative potency of degraded PhtD protein is reduced progressively with the storage time point. However, this reduction in potency was not as significant as for PcpA and did not reflect the antigenicity results obtained with the ELISA data.

A pool of anti-PhtD monoclonal antibodies (3C, 4D5, 5B7, 6B7, 8G4 and 8H6) was used at a final concentration of 2.5 μg/mL each (using PBS as diluent) in a competitive SASSY as described herein (FIG. 4, Table 6). Diluted monoclonal antibodies were preincubated for at least 1 hour at room temperature with a titration (7:10 dilution factor starting at 10 μg/mL final concentration) of either intact or stressed PhtD proteins.

TABLE 6
PhtD Relative Potency-Monoclonal

| Relative potency | Relative | Relative | Relative |
|---|---|---|---|
| 0 wk | potency 1 wk | potency 4 wk | potency 0 wk |
| 1 | 0.92 | 0.89 | 0.83 |

### Example 3

### Correlation Between SASSY and Passive Protection

Correlation between SASSY and Passive Protection using Polyclonal Sera from Rabbit Immunized with Intact versus Degraded monovalant PcpA Formulations

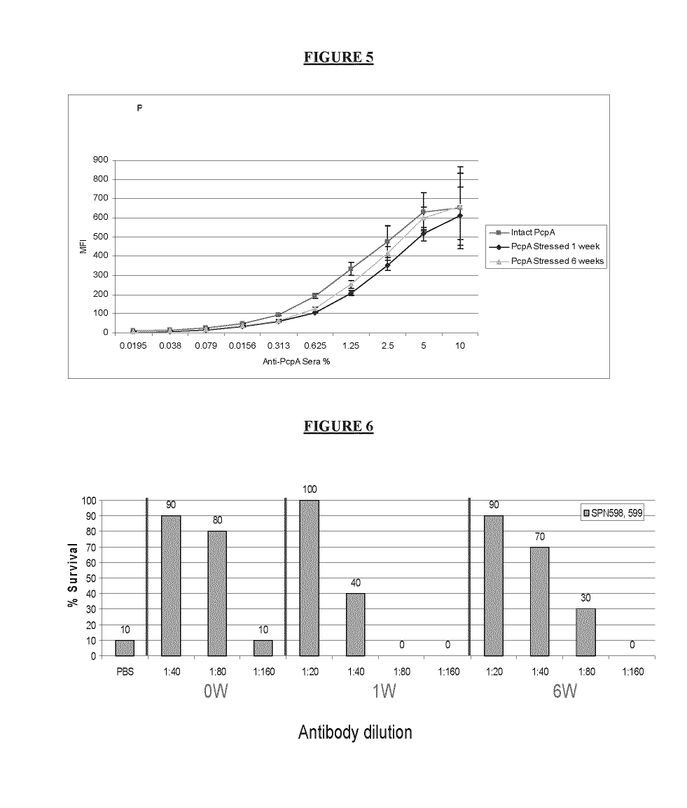

A study was conducted to evaluate the relation of the competitive SASSY and antigenicity results to the actual biological effect of degraded proteins in animals Rabbits were immunized IM with either intact or degraded protein as described above and sera was collected following two boosts and used for IgG determination, SASSY and passive protection. While no significant differences were detected in the PcpA formulations treated for 6 weeks at 43-47° C., rabbits immunized with PcpA degraded protein for one week showed a 2 fold drop in anti-PcpA antibody titer and a 4 fold drop in passive protection. In order to verify whether the SASSY assay can detect these qualitative differences the same sera was also tested for their binding to A66.1 in SASSY assay. Results have also indicated a 30% drop in MFI in comparison to immune responses generated with intact PcpA protein, while a less notable drop was determine with the 6 weeks stressed samples. These results follow the same trend observed in the passive protection studies, suggesting that the SASSY is a measure of the amount of antibodies bound to the bacteria and the breadth of the response generated. On the other hand, the IgG titer determination alone was not sufficient to distinguish differences between these samples. These results are summarized in Table 7, while the SASSY results are shown in FIG. 5 and the passive protection results are shown in FIG. 6.

TABLE 7
Summary of SASSY, Passive Protection and anti-PcpA IgG titer

| | SASSY (potency | Passive Protection | IgG titer |
|---|---|---|---|
| | relative to intact | (Highest Protective | (end point |
| PcpA Protein | protein) | Dilution) | dilution) |
| 0 week (intact) | 1 | 1:80 | 36400 |
| 1 week | 0.75 | 1:20 | 12800 |
| 6 week | 0.87 | 1:40 | 19200 |

Correlation Between SASSY and Passive Protection Uusing Polyclonal Sera from Rabbit Immunized with Intact Versus Degraded Monovalent PhtD Formulations

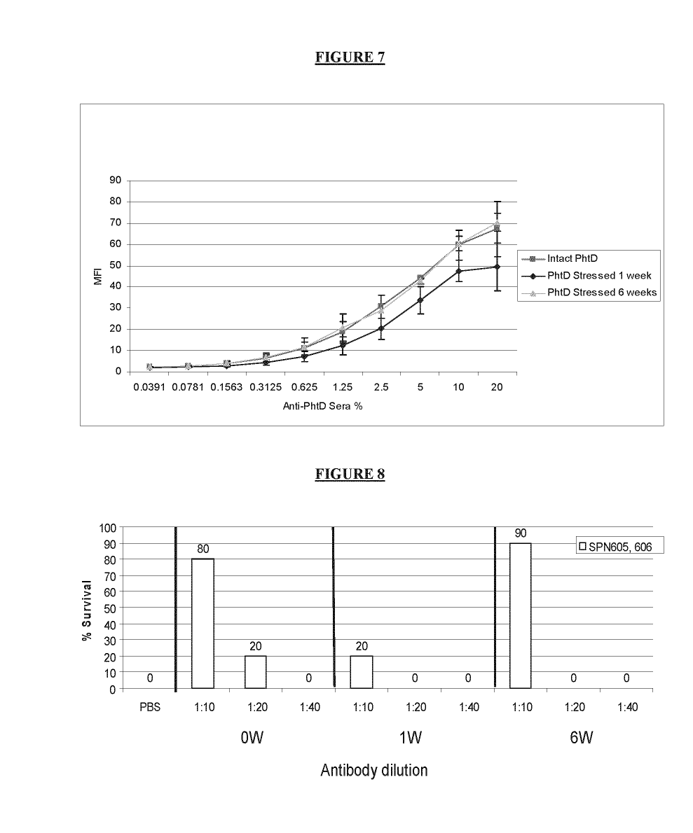

The same analysis that was performed for PcpA degraded samples was also applied for PhtD degraded samples. Interestingly the same conclusions were also drawn from PhtD degraded samples. The greater drop in potency was associated with storage at 43-47° C. for 1 week, while longer incubations led to recovery of potency both detected by SASSY and passive protection. As shown in FIGS. 7 and 8, 1 week stressed samples have a 25% drop in the MFI and lose their protective ability in vivo. Interestingly, quantification of the amount of antibodies generated did not follow the same trend, confirming again that protection depends on the presence of functional antibodies and the amount of antibodies rather then only the amount of antibodies generated. Interestingly, the SASSY assay can measure those two attributes as illustrated in the results generated. Table 8 summarizes the results for SASSY, passive protection and IgG titers.

TABLE 8
Summary of SASSY Passive Protection and anti-PhtD IgG titers

| | SASSY (potency | Passive Protection | Anti-PhtD IgG |
|---|---|---|---|
| | relative to intact | (Highest Protective | titers (end point |
| PhtD Protein | protein) | Dilution) | dilution) |
| 0 week (intact) | 1 | 1:10 | 19200 |
| 1 week | 0.76 | 0 | 12800 |
| 6 week | 1.0 | 1:10 | 57600 |

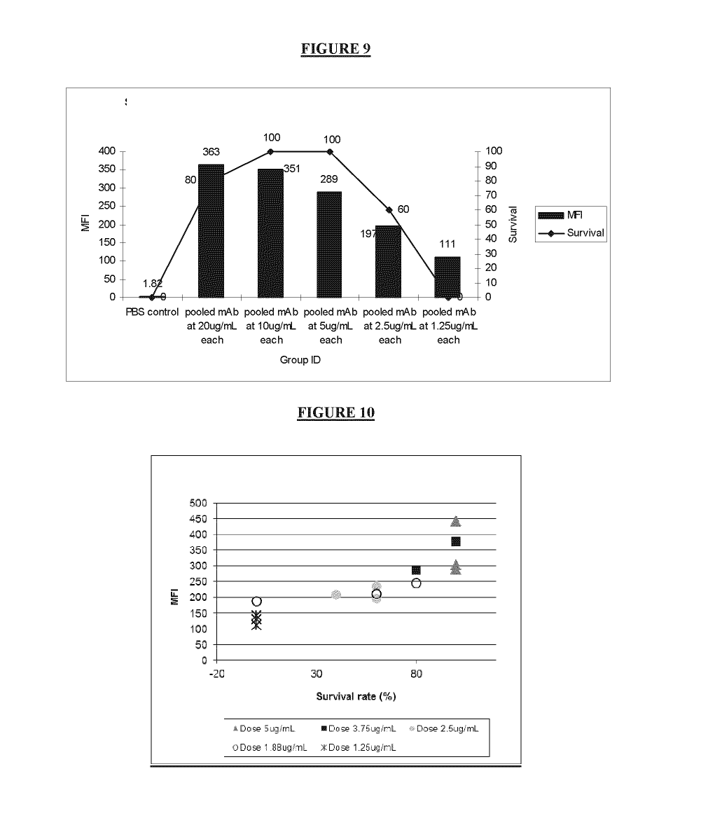

In order to establish a correlation between passive protection and competitive SASSY, we wanted to ensure first that binding of functional monoclonal antibodies to the bacteria leads to protection in the in vivo animal model. This could then result in the replacement of in vivo testing with a MFI measurement of the antibody binding by flow cytometry allowing the implementation of an all in vitro assay to assess potency of both PcpA and PhtD. Given that a pool of 5 monoclonal antibodies specific for PcpA were shown to be stability indicating (1-12, 1-29, 2B3, 6A4, and 9G11) in the competitive in vitro assay, the same pool was assessed for its direct binding to S. pneumoniae A66.1 strain in SASSY and its potential to protect following IV challenge of CBA/N mice. Results as shown in FIG. 9 indicated a dose response observed in both SASSY and passive protection. A66.1 strain pre-incubated with a pool of monoclonal antibodies at a concentration of 10 μg/mL is saturating in terms of binding to A66.1 (MFI=351) and provides optimum protection in mice, while lower concentration of monoclonal antibodies (2.5 μg/mL) provide a suboptimum binding to A66.1 (MFI=197) and only 60% survival.

It was determined that an MFI equal or lower than 111 is no longer protective. Four independent preparations of A66.1 pre-incubated with antibodies were performed and a correlation between SASSY and passive protection was assessed using Spearman Coefficient. Both FIG. 10 and Table 9 indicate a very strong correlation between both the in vitro and in vivo assay with an R²=0.963.

TABLE 9
Statistical Analysis - Spearman Correlation of Competitive SASSY and
Passive Protection Studies using anti-PcpA Monoclonal Antibodies
Spearman Correlation Coefficients
Prob > |r| under H0: Rho = 0
Number of Observations

| | dose | survival | sassy |
|---|---|---|---|
| dose | 1.00000 | 0.88944 | 0.88378 |
| | | <.0001 | <.0001 |
| | 15 | 14 | 15 |
| survival | 0.88944 | 1.00000 | 0.96310 |
| | <.0001 | | <.0001 |
| | 14 | 14 | 14 |
| sassy | 0.88378 | 0.96310 | 1.00000 |
| | <.0001 | <.0001 | |
| | 15 | 14 | 15 |

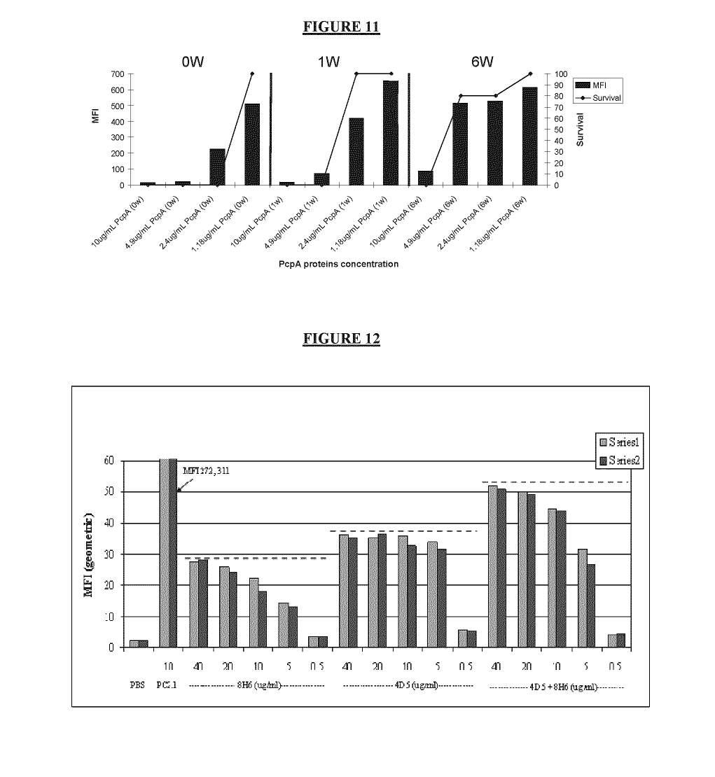

Since a correlation was achieved between the two assays, degraded PcpA proteins were tested in both the competitive SASSY and passive protection in order to determine whether the competitive SASSY can also be linked to a functional read out. In this experiment, degraded proteins as described above were first incubated with monoclonal antibodies at room temperature as described herein and A66.1 fresh culture was added to each sample to monitor both survival in mice and binding by detecting the MFI. Results as shown in FIG. 11, indicate a significant reduction of both MFI and survival when intact PcpA protein was added at 10, 4.9 and 2.4 μg/mL of protein, while this is only observed at 10 and 4.9 μg/mL with the one week degraded PcpA and only 10 μg/mL with the six weeks degraded PcpA. These results demonstrate that the in vitro competitive SASSY can be used to assess the biological activity and stability of PcpA formulations without having to use animal models.

Monovalent PcpA formulated with AIOOH stored for one week at 43-47° C. demonstrated a slight reduction in the protein content however a much larger impact was observed in terms of its antigenicity and potency as assessed by the in vitro or the in vivo assays. Interestingly, the drop in IgG titers observed in rabbits immunized with one week degraded samples may not be considered significant (≧4 fold difference is considered biologically relevant). In contrast, the protective ability of the sera as tested in passive protection showed a significant drop in comparison to intact protein (4-fold drop: 1/80 compared to 1/20 dilution provide protection) suggesting that even though the protein was able to generate antibodies the protective epitopes may have been masked due to conformational changes in the protein or tighter interactions between the protein and the adjuvant. This may have lead to a reduced amount of functional antibodies and ultimately the reduction in potency. These results indicate that both the qualitative and quantitative aspect of the humoral immune response generated against PcpA should be considered as a measure of potency.

In the studies performed here both the competitive SASSY and antigenicity ELISA were able to detect the loss of protective epitopes in the drug product when subjected to high temperature storage. Interestingly, even though the antigenic ELISA and the in vitro competitive SASSY indicated a significant drop in PcpA antigenicity following 6 weeks storage at 43-47° C. using monoclonal antibodies, passive protection results have showed that those degraded samples can still generate protective immune responses. This could be explained by the fact that these proteins are completely degraded to linear peptides (confirmed by SD S-PAGE) which would include protective epitopes. These epitopes can then become accessible for immune recognition and generation of functional antibody responses. In several models, a correlation between SASSY or competitive SASSY with passive protection was demonstrated indicating that protection in mice is mediated by antibodies binding to the bacteria as the initial step. It is unknown how the bacteria is cleared in mice. In terms of PhtD stressed material, the antigenicity ELISA and the passive protection studies using sera from immunized rabbits were able to detect a difference with the samples treated for 1 week at 43-47° C. compared to intact protein. Furthermore, a direct SASSY performed on the same sera samples from immunized rabbits showed the same trend as passive protection, indicating that both assays are aligned. Pre-incubation of A66.1 or WU2 with the same pool of monoclonal antibodies that were used in the competitive SASSY were not protective in mice, and therefore a correlation between SASSY and passive protection was demonstrated.

### Example 4

A. Detection of Repertoire Expansion Using SASSY

To determine whether SASSY could be used to detect repertoire expansion, two monoclonal antibodies were utilized alone and simultaneously to determine MIF. The results are demonstrated in FIG. 12. As shown therein, an antibody epitope repertoire expansion may be be detected as an increase in surface binding (MFI difference) using SASSY. FIG. 12 illustrates duplicate points (series 1 and 2) and is representative of three independent studies.

B. SASSY Using Sera of Vaccinated Human Beings

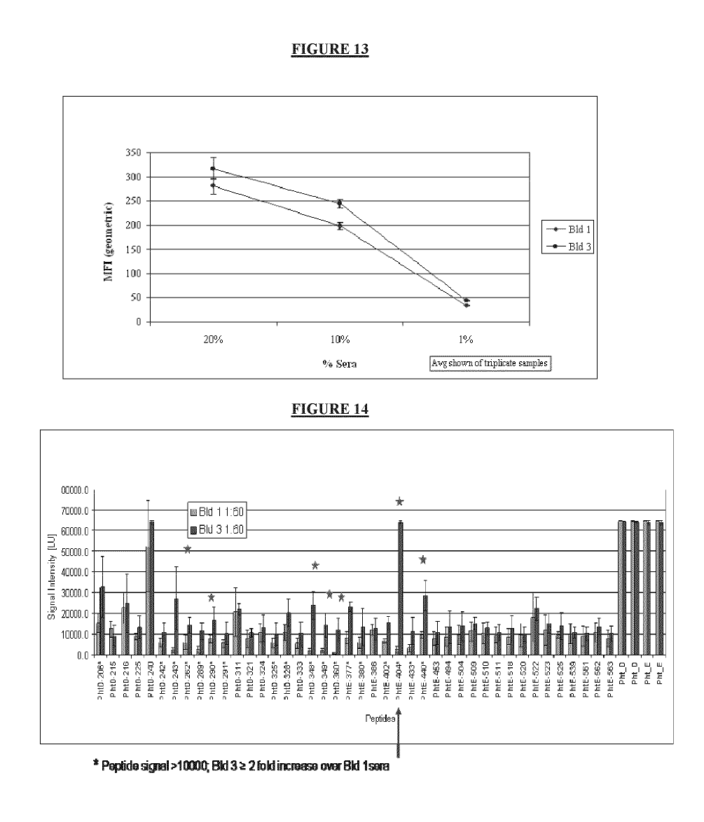

Experiments were also carried out to determine whether SASSY results would correlate with in vivo immunization studies in humans Sera from human beings enrolled in a clinical trial and known to contain functional, PhtD-specific antibodies were tested in SASSY. Post-vaccine sera (Bld3) saturated at a higher MFI than the pre-vaccine serum (Bld1) when three of three sera sample pairs were tested (#37, #43 and #61). A representative plot for #37 is shown in FIG. 13. It is noted that a negative control sera pair from the subject with weak PhtD antibody titre increase (#21) did not show an increase in MFI.

C. Unique Peptide Epitopes

It was also determined that the immune responses observed in the same clinical trial were reactive against unique epitopes. As shown in FIG. 14, bleed 1 (Bld1) vs. bleen 3 (Bld3) sera from subject #37 were screened for binding to 15-mer overlapping peptides spanning PhtD and PhtE proteins using the ProArray peptide microarray (Prolmmune). A positive signal was considered to have at least 10 K LU and only pairs with greater than 10 K LU are shown. Significant difference is considered to have at least a 3-fold increase (star) indicating a potentially new antibody epitope recognition in Bld3. As shown therein, new epitopes included PhtD-262 (GVAVPHGNHYHFIPY (SEQ ID NO:1), PhtD-290 (FYNKAYDLLARIHQD (SEQ ID NO:2), PhtD-348 SADNLYKPSTDTEET (SEQ ID NO:3), PhtD-349 (YKPSTDTEETEEEAE (SEQ ID NO:4), PhtD-360 (ETLTGLKSSLLLGTK (SEQ ID NO: 5), PhtE-404 (YIPKSDLSASELAAA (SEQ ID NO:6), and PhtE-440 (AYIVRHGDHFHYIPK (SEQ ID NO:7). It was observed that PhtE-404, and PhtE-440 were also cross-reactive with PhtD.

While the present invention has been described in terms of the preferred embodiments, it is understood that variations and modifications will occur to those skilled in the art. Therefore, it is intended that the appended claims cover all such equivalent variations that come within the scope of the invention as claimed

1. A method comprising the steps of:
a) contacting a drug product comprising one or more antigens with an antibody composition comprising antibodies reactive against at least one of the one or more antigens to produce a test composition;
b) contacting the test composition with a test cell expressing at least one of the antigens; and,
c) detecting the binding of antibodies to the test cell.

2. The method of claim 1 wherein the antigen is a cell surface antigen.

3. The method of claim 1 wherein the test cell is selected from the group consisting of a microorganism, tumor cell, cell expressing a viral antigen, or a recombinant cell.

4. The method of any one of claims 1-3 wherein the antibody composition is selected from the group consisting of serum, ascites, cell culture supernatant, polyclonal antisera, a monoclonal antibody composition, and mixtures thereof.

5. The method of any one of claims 1-4 wherein the microorganism is in solution or affixed to a solid surface.

6. The method of any one of claims 1-5 wherein the detection of antibodies on the microorganism is by flow cytometry.

7. The method of any one of claims 1-6 wherein the detection of antibodies on the test cell indicates the drug product is not intact.

8. The method of any one of claims 1-6 wherein a lack of detection of antibodies on the test cell indicates the drug product is intact.

9. The method of claim 4 wherein the serum is derived from a mammal immunized with an antigen present in the drug product.

10. The method of any one of claims 1-9 wherein the antigen comprises at least one conformational epitope reactive with the antibodies.

11. The method of any one of claims 1-10 further comprising separately carrying out steps (a)-(c) with first and second drug products and comparing the amount of antibody detected on the test cell for the first and second drug products.

12. The method of claim 11 wherein the first drug product is a control drug product.

13. The method of claim 11 wherein the detection of more antibody following incubation with the second drug product as compared to the first drug product indicates that second drug product is less intact or not intact relative to the first drug product.

14. The method of claim 11 or 13 wherein the first and second drug product are different lots of the same drug product.

15. The method of any one of claims 11-14 wherein drug product potency is determined.

16. The method of claim 15 wherein the drug product is a vaccine.

17. The method of any one of claims 1-14 wherein the drug product is a vaccine.

18. A kit for carrying out a method of any one of claims 1-15 comprising components required to carry out a control reaction and instructions for use.

19. A method for determining vaccine efficacy in a mammal comprising contacting mammalian sera with a test cell expressing at least one cell surface antigen with which the mammal was previously vaccinated; and, detecting the binding of antibodies of the sera, if present therein, to the test cell.

20. The method of claim 19 further comprising comparing the binding of antibodies of a first sera sample of a mammal to whom the vaccine was not previously administered to the binding of antibodies from a second sera sample of the mammal following administration of the vaccine.

21. The method of claim 20 wherein the second sera sample is obtained from the mammal at least seven days following administration of the vaccine.

22. The method of any one of claims 19-21 wherein the mammal is a human being.

23. The method of any one of claims 19-22 wherein the test cell is selected from the group consisting of a microorganism, tumor cell, cell expressing a viral antigen, or a recombinant cell.

24. A peptide selected from the group consisting of PhtD-262 (GVAVPHGNHYHFIPY (SEQ ID NO:1), PhtD-290 (FYNKAYDLLARIHQD (SEQ ID NO:2), PhtD-348 SADNLYKPSTDTEET (SEQ ID NO:3), PhtD-349 (YKPSTDTEETEEEAE (SEQ ID NO:4), PhtD-360 (ETLTGLKSSLLLGTK (SEQ ID NO: 5), PhtE-404 (YIPKSDLSASELAAA (SEQ ID NO:6), and PhtE-440 (AYIVRHGDHFHYIPK (SEQ ID NO:7).

25. A composition comprising a peptide of claim 24.


SEQUENCE LISTING (7 sequences)

SEQ ID NO: 1 - protein, 15 aa, Streptococcus pneumoniae
GVAVPHGNHYHFIPY

SEQ ID NO: 2 - protein, 15 aa, Streptococcus pneumoniae
FYNKAYDLLARIHQD

SEQ ID NO: 3 - protein, 15 aa, Streptococcus pneumoniae
SADNLYKPSTDTEET

SEQ ID NO: 4 - protein, 15 aa, Streptococcus pneumoniae
YKPSTDTEETEEEAE

SEQ ID NO: 5 - protein, 15 aa, Streptococcus pneumoniae
ETLTGLKSSLLLGTK

SEQ ID NO: 6 - protein, 15 aa, Streptococcus pneumoniae
YIPKSDLSASELAAA

SEQ ID NO: 7 - protein, 15 aa, Streptococcus pneumoniae
AYIVRHGDHFHYIPK